(19) United States
(12) Patent Application Publication
(10) Pub. No.: US 2023/0326584 A1
(43) Pub. Date: Oct. 12, 2023

(54) SYSTEM AND METHOD FOR SCHEDULING HEALTHCARE-RELATED SERVICES

(71) Applicant: THE I.V. DOC INC., Watermill, NY (US)

(72) Inventor: Adam Nadelson, Watermill, NY (US)

(73) Assignee: THE I.V. DOC INC., Watermill, NY (US)

(21) Appl. No.: 17/934,197

(22) Filed: Sep. 21, 2022

Related U.S. Application Data
(60) Provisional application No. 63/329,862, filed on Apr. 11, 2022.

(51) Int. Cl. G16H 40/20; G16H 80/00; G16H 20/00

(52) U.S. Cl. CPC G16H 40/20; G16H 20/00; G16H 80/00

(57) ABSTRACT

A system and method for scheduling healthcare-related services. The method includes receiving identifying data associated with a user. The method further includes presenting service(s) available to the user based on the data associated with the user. The method also includes receiving a selection of the service(s) presented to the user. The method further includes sending a service request message to one or more clinicians based on the received identifying data and the selection of the service(s). The method additionally includes receiving a confirmation message indicating a physician's confirmation to provide the service(s). The method also includes scheduling a time and location for the clinician to provide the service(s).

### BACKGROUND

The healthcare industry is undergoing a meaningful transformation built upon an underlying digital foundation. This digital foundation paves the way for a digital healthcare journey for patients, clinicians, and healthcare organizations. One of the last things a patient wants to do when feeling unwell, distressed, dehydrated, exhausted, etc. is to travel to a medical office only to endure long wait times and unnecessary exposure to various ailments and infections. This hinders the ability of busy, successful, and on-the-go people to address conditions like the flu, food poisoning, severe hangover, and other wellness issues promptly and effectively in the privacy of their home or current location. Such people may resort to inefficient oral remedies or not visit a medical facility at all.

This can be avoided when clinicians travel to a patient's location and provide healthcare services on-site, whether it be in the comfort of the patient's home, office, hotel room, etc. On-site healthcare service also provides the opportunity for treatment to be administered discreetly and kept in the strictest confidence.

### SUMMARY

A need exists for a digital platform that can connect patients with qualified and licensed clinicians who can provide on-site healthcare services.

In general, in one aspect, embodiments relate to a method for scheduling healthcare-related treatments. The method includes receiving patient-identifying data from a first device (e.g., one that belongs to a patient), where the patient-identifying information includes a name, a date of birth, and a phone number. The method further includes receiving a selection of at least one treatment, a location for the treatment, and a time for the treatment from the first device. The method also includes transmitting treatment request information about the patient to a second device (e.g., one that belongs to a nurse). The treatment request information may include the location for a treatment. The method further includes receiving an acceptance of the treatment from the second device. The method additionally includes transmitting information about the treatment to a third device (e.g., one that belongs to a physician). The method also receives an approval of the treatment from the third device. The method may transmit acceptance of the treatment to the first device. In addition, the method may transmit additional information about the patient to the second device in response to the approval of the treatment.

In another aspect, embodiments relate to a system for scheduling healthcare-related treatments. The system may include a computer processor, a memory, and a treatment scheduling engine executing on the computer processor. The engine may be configured to receive patient-identifying data from a first device (e.g., one that belongs to a patient), where the patient-identifying information includes a name, a date of birth, and a phone number. The engine may further be configured to receive a selection of at least one treatment, a location for the treatment, and a time for the treatment from the first device. The engine may also be configured to transmit treatment request information about the patient to a second device (e.g., one that belongs to a nurse). The treatment request information may include the location for a treatment. The engine may also be configured to receive the acceptance of the treatment from the second device. The engine additionally may also be configured to transmit information about the treatment to a third device (e.g., one that belongs to a physician). The engine is additionally configured to receive an approval of the treatment from the third device. The engine may also be configured to transmit acceptance of the treatment to the first device. In addition, the engine may be configured to transmit additional information about the patient to the second device in response to the approval of the treatment.

### BRIEF DESCRIPTION OF THE DRAWINGS

Embodiments are illustrated by way of example, and not by way of limitation, in the figures of the accompanying drawings and in which like reference numerals refer to similar elements.

FIG. 1 shows a schematic diagram of a system, in accordance with one or more embodiments.

FIG. 2 shows an exemplary screenshot of a user interface for providing patient-identifying information, in accordance with one or more embodiments.

FIG. 3 shows an exemplary screenshot of a user interface for selecting a health-related service on the healthcare platform

FIG. 4 shows an exemplary screenshot of a user interface presented to a clinician.

FIG. 5 shows an exemplary medical diagnosis form to be completed by a physician.

FIG. 6 illustrates a flowchart of an exemplary process, in accordance with one or more embodiments.

FIG. 7 illustrates a flowchart of an exemplary process, in accordance with one or more embodiments of the invention.

FIGS. 8 and 9 show a computing system and network architecture in accordance with one or more embodiments.

### DETAILED DESCRIPTION

A portion of the disclosure of this patent document may contain material that is subject to copyright protection. The copyright owner has no objection to the facsimile reproduction by anyone of the patent document or the patent disclosure, as it may appear in the Patent and Trademark Office patent file or records, but otherwise reserves all copyrights whatsoever.

Specific embodiments will now be described in detail with reference to the accompanying figures. Like elements in the various figures are denoted by like reference numerals for consistency. In the following detailed description of embodiments, numerous specific details are set forth in order to provide a more thorough understanding of the invention. While described in conjunction with these embodiments, it will be understood that they are not intended to limit the disclosure to these embodiments. On the contrary, the disclosure is intended to cover alternatives, modifications and equivalents, which may be included within the spirit and scope of the disclosure as defined by the appended claims. It will be apparent to one of ordinary skill in the art that the invention can be practiced without these specific details. In other instances, well-known features have not been described in detail to avoid unnecessarily complicating the description.

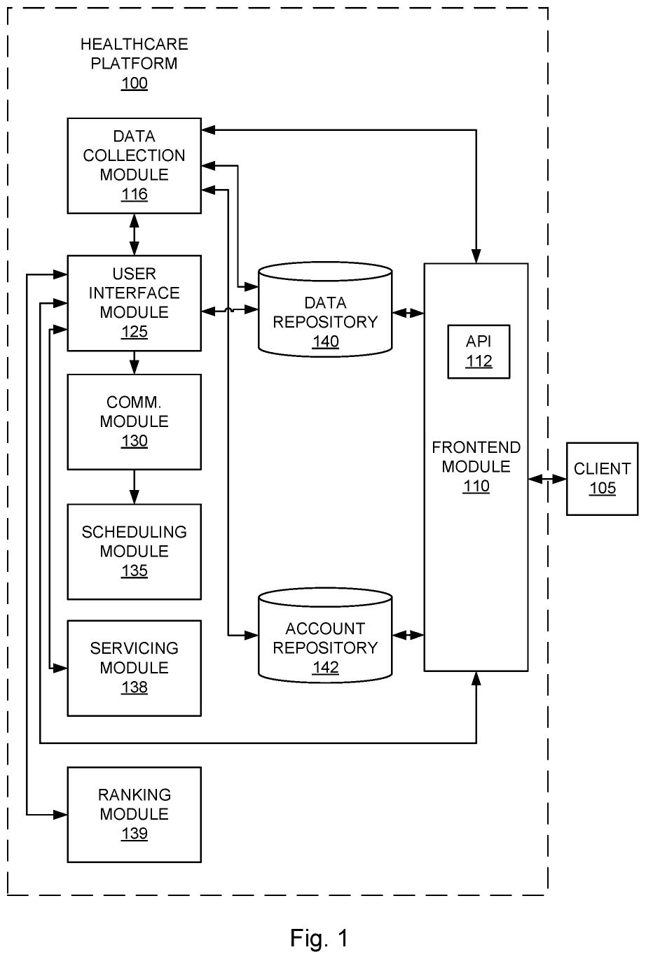

FIG. 1 shows a healthcare platform 100 and a client computing device 105 in accordance with one or more embodiments. As shown in FIG. 1, healthcare platform 100 has multiple components including a frontend module 110 with an application programming interface (API) 112, a data collection module 116, a user interface module 125, a communication module 130 (operable to be HIPAA-compliant/satisfy HIPAA communication requirements), a scheduling module 135, a servicing module 138, a ranking module 139, a data repository 140, and an account repository 142.

In one or more embodiments, the healthcare platform 100 is a platform for matching and facilitating healthcare/lifestyle services between users (e.g., patients) and clinicians/practitioners (e.g., nurses, physician's assistants, doctors/physicians, or other qualified healthcare professional). For example, the healthcare platform 100 may facilitate the scheduling of on-site/mobile intravenous therapy services (e.g., for a dehydrated patient). It should be understood that the terms “user” and “patient” may be used interchangeably herein, and that the terms “clinician” and “practitioner” may be used interchangeably herein. In some embodiments described herein, a clinician may visit multiple patients in a day—each at their own homes (or a location that's not necessarily a clinic or hospital). Also, herein the term “provider” may refer to any person or entity that provides medical care or treatment, including clinicians/practitioners, Doctors of Medicine, Doctors of Osteopathic Medicine, Physician Assistants/Associates, Nurse Practitioners, Psychologists, Medical Assistants, therapists, social workers, health insurance plans, urgent care clinics, health clinics, hospitals, etc.

The healthcare platform 100 may include Electronic Medical Record (EMR) data, Electronic Health Record (EHR) data, History of Present Illness (HPI) data (collectively referred to as “EMR” data herein), Examination Assessment data, and/or Diagnosis & Plan data. In some embodiments, the healthcare platform 100 is an EMR/EHR/HPI system and/or Examination Assessment and/or Diagnosis & Plan system. The healthcare platform 100 may include EMR data for patients corresponding to different geographies (e.g., ZIP codes, cities, counties, states, regions, etc.).

In some embodiments, the EMR data is organizable and accessible according to geography. For example, the EMR data may be categorized and siloed/separated based on geographic location of the patient or services having been/being/to be rendered. In one example, the healthcare platform 100 may include EMR data for a patient in San Francisco, and also EMR data for the same patient but in New York. In some embodiments, clinicians in San Francisco can access the patient's EMR data of New York, while clinicians in New York can access the patient's EMR data of San Francisco. In some embodiments, a patient in San Francisco can access the patient's account for managing services rendered in New York (e.g., including billing information).

In some embodiments, a patient's EMR data corresponds to primarily a single account, however, the accessible EMR data is limited to clinicians in respective service geographies. In another embodiment, the patient's EMR data is distributed amongst multiple service geographies and standalone/independent in each distributed geography, meanwhile the independent distributed EMR data is linked/associated with the EMR data of other geographies. For example, the healthcare platform 100 may include EMR data for a patient in San Francisco that is standalone, New York that is standalone, and Dallas that is standalone. Meanwhile, in some embodiments, the San Francisco, New York, and Dallas EMR data is linked. That way, a patient may visit multiple locations that are all up to date with the patient's EMR or account data. For example, if the patient travels from San Francisco to New York, the New York clinician(s) will have access to the most recent EMR data to provide better care. In another example, the patient's payment information (e.g., credit card) is readily available for remitting services rendered in New York (and any other siloed regions). As will be described below, in some cases the payment for a treatment may cost the same patient a different amount if that patient is receiving service from a provider in San Francisco, New York, or Dallas.

The healthcare platform 100 may be configured to enable users to communicate in “real-time” with a clinician or physician, i.e., to converse with them with a minimal delay. In other words, the healthcare platform 100 may allow a user to submit messages and may display the messages to one or more other users (e.g., clinicians or administrative staff) within a reasonable time frame to facilitate a live conversation between the users. As an example, a patient may request a treatment, and before a clinician accepts the treatment, the clinician may have a real-time conversation with the patient. This real-time conversation may cost a patient money, or change a patient or clinician's rating (as discussed below).

The data collection module 116 may be configured to collect data about a user of the healthcare platform 100. The data may include healthcare-related information about the user. For example, the data collection module 116 may collect a user's vital information such as heart rate, temperature, blood pressure, blood oxygen level, etc. Such data may be stored in the data repository 140. In some embodiments, healthcare platform 100 may provide a user with questions about their health (e.g., whether the user has any allergies to any medications) at the user's electronic device (e.g., client 105). Responses to these questions may then be sent back to healthcare platform 100, and may be used to determine a price of a treatment. Responses to these questions may also be added to a patient's EMR, clinicians, and physicians.

The user interface module 125 may be configured to render a user interface usable by a user, clinician, or physician. The user interface may be displayed on client device 105, which may be an electronic device. In some embodiments, at least three different types of accounts may exist. For example, a first electronic device may be logged into a patient account, a second electronic device may be logged into a clinician account, and/or a third electronic device may be logged into a physician account.

The user interface module 125 may be configured to facilitate the communication of messages of the healthcare platform 100 (e.g., between patients, clinicians, etc.). The user interface module 125 may be configured to be HIPAA-compliant and/or satisfy HIPAA communication requirements.

In addition or in place, the communication module 130 may be configured to facilitate the communication of messages of, to, and from the healthcare platform 100. For example, the communication module 130 may be configured to transmit and receive text messages (e.g., cellular phone SMS or MMS), short code messages, or internet-based notification (iOS app or mobile web notification) to and from client 105. The communication module 130 may be configured to be HIPAA-compliant (e.g., secured and/or password-protected links store patient health information within the healthcare platform 100). Both the user interface module 125 and the communication module 130 may be communicate the same, similar, or different messages.

The scheduling module 135 may be configured to facilitate the scheduling of a healthcare related service requested by the user via the healthcare platform 100.

The servicing module 138 may be configured to receive a treatment request for a healthcare-related service from a user. The treatment request may be received, for example, via client 105. It should be understood that, herein, the terms “service” and “treatment” may be used interchangeably. Similarly, a “service request” and a “treatment request” may be used interchangeably.

The ranking module 139 may be configured to generate a ranked list including treatment requests received by the servicing module 138. The ranking module 139 may rank a received service request amongst other receiving service requests at a previous time. The ranking may be generated based at least in part on identifying data associated with the patient collected by the data collection module 116.

The healthcare platform 100 may also include a pricing module (not shown) configured to determine a service fee for healthcare-related treatments offered by the healthcare platform 100. All or a portion of the service fee may be earned by the clinician that performs the healthcare-related service requested by the user. In some embodiments, the service fee determined by the pricing module (not shown) may be based at least in part on a total number of healthcare-related service requests pending on the healthcare platform 100. By way of example, a pending treatment request may be a treatment request received by the servicing module 138 that has not yet been matched with a providing clinician for performing the service, or, a pending service request may be a service request that has been matched but has not yet been performed/carried out. In some embodiments, matching a clinician with a patient/user may mean that a particular treatment request has been sent to a clinician but not yet accepted by a clinician, while in other embodiments, matching a clinician with a patient/user may mean that a treatment request has been accepted by a particular clinician.

Although the components of the healthcare platform 100 are depicted as being directly communicatively coupled to one another, this is not necessarily the case. For example, one or more of the components of the healthcare system 100 may be communicatively coupled via a distributed computing system, a cloud computing system, or a networked computer system communicating via the Internet.

Although only one healthcare platform 100 is illustrated, it should be appreciated that this one healthcare platform may represent many healthcare platforms, arranged in a central or distributed fashion. For example, such healthcare platform may be organized as a central cloud and/or may be distributed geographically or logically to edges of a system such as a content delivery network or other arrangement. It is understood that virtually any number of intermediary networking devices, such as switches, routers, servers, etc., may be used to facilitate communication.

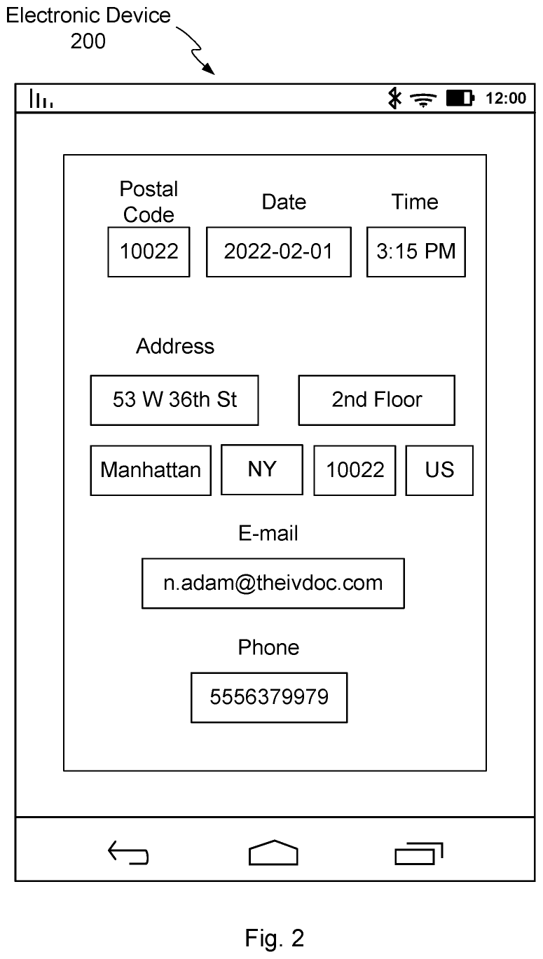

FIG. 2. shows an exemplary screenshot of a user interface for a user intake form on the healthcare platform 100 (FIG. 1). The example shown in FIG. 2 may be generated by the user interface module 125 (FIG. 1). A user may log in to the healthcare platform 100 using his/her credentials associated with the healthcare platform 100. If this is the user's first-time using healthcare platform 100, the user may be presented with the option to create an account. The account information may be stored in the account repository 142 (FIG. 1).

Upon logging in, the user may be asked to provide user identifying information, such as his/her ZIP code, a desired location for a requested healthcare service to be performed, and personal contact information. They may also be asked to specify any provider (e.g., clinician or physician) they would prefer to work with.

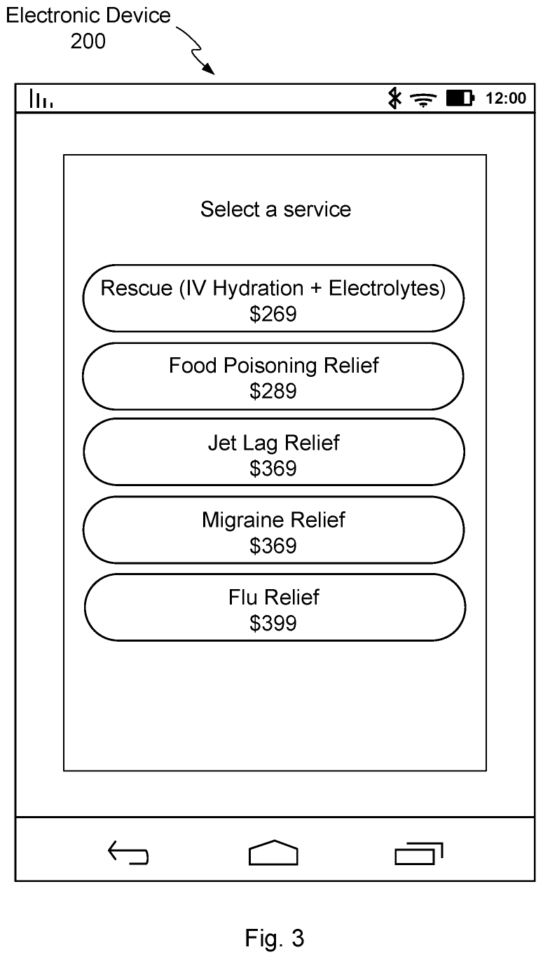

FIG. 3 shows an exemplary screenshot of a user interface for selecting a treatment on the healthcare platform 100 (FIG. 1). The example shown in FIG. 3 may be generated by the user interface module 125 (FIG. 1) and presented to the user via client 105 (FIG. 1). The health-related services presented to the user may be based at least in part on the user identifying information. For example, certain health-related services may only be available in certain ZIP codes. As such, the health-related services presented to the user in FIG. 3 may be only those that are available to the user based on the user's inputted ZIP code depicted in FIG. 2.

In this example, the user is presented with five different health-related services available for selection. For example, the user is presented with (1) Rescue (IV Hydration+Electrolytes+Medications for symptoms [e.g., selected by the physician]) for $269, (2) Food Poisoning Relief for $289, (3) Jet Lag Relief for $369, (4) Migraine Relief for $369, and (5) Flu Relief for $399.

Selection by the physician may be suggested by healthcare platform 100. In some embodiments, a physician may be incentivized to work on service requests and provide treatment. For example, to provide certain medications/amounts of medications based on the cost of the treatment or the medications, provided it meets standards of care. In another example, healthcare platform 100 may pay a physician more if they were to approve treatments and medication that are within a threshold amount of a projected cost of a treatment (e.g., the projected cost may be the cost shown to a patient when they made the treatment request), provided it meets standards of care. This may cause patients to trust the system more because it causes an estimated amount of service to be correct more often (if the system does provide an estimated amount of service, which it may not when it is providing a total/complete cost of service). Physicians may be incentivized in other ways. For example, a physician may receive additional compensation based on how quickly they approve treatments (meeting the standard of care), how many treatments they approve per day (meeting the standard of care), etc.

The user may then select one or more treatments of interest via a user interface of the client 105 (FIG. 1). Additionally, after the user selects one or more treatments of interest, the user interface may display a health questionnaire requesting information pertaining to present and/or historical health data associated with the user.

The answers to the questionnaire and/or the present and/or historical health data associated with the user may be transmitted, via the communication module 130 (FIG. 1), in an appointment request to a clinician (e.g., typically a nurse or a physician's assistant, but in some embodiments a doctor/physician or other qualified healthcare professional) associated with the healthcare platform 100. In addition to the present and/or historical health data, the appointment request may also include Electronic Medical Record (EMR) data, Electronic Health Record (EHR) data, History of Present Illness (HPI) data, Examination Assessment data, and/or Diagnosis & Plan data to the clinician.

In some embodiments, EMR, EHR, HPI, Examination Assessment data, and/or Diagnosis & Plan data may have billing data associated with it in a manner such that the clinician is able to view the billing data. In other embodiments, the clinician is not able to view the billing data.

In some embodiments, after the user submits the request, the healthcare platform 100 collects payment (or a payment hold like a credit card hold) for the services to be rendered. In some embodiments, a request notification associated with the transmission is provided to practitioners on priority and/or then to practitioners not off call (e.g., all available nurses).

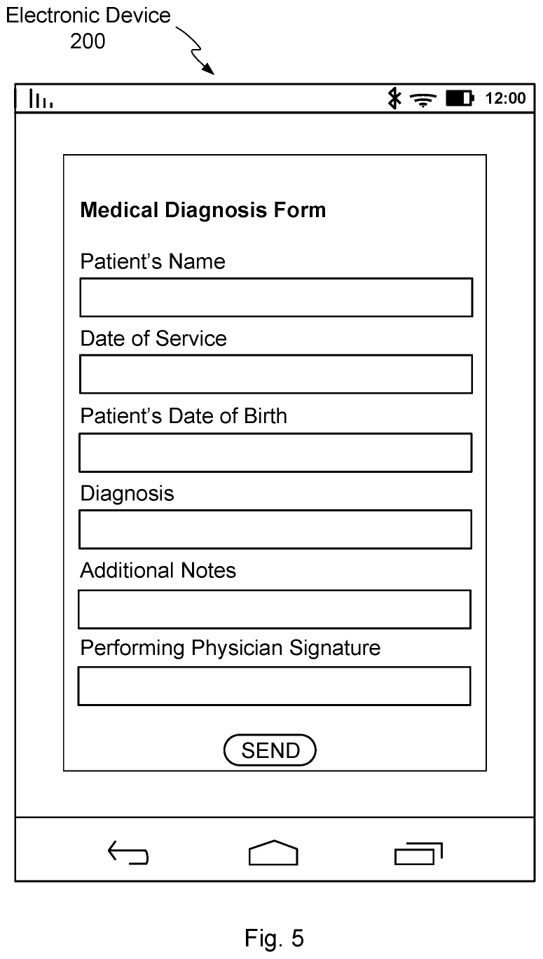

After the practitioner reviews the various data transmitted by the communication module 130 (FIG. 1), the practitioner may approve the healthcare-related treatment(s) requested by the user on the healthcare platform 100. The practitioner's approval of the healthcare-related treatment may be captured in a medical diagnosis form, similar to the one depicted in FIG. 5. If the practitioner approves the treatment(s), a notification associated with the pending service request may be routed to one or more physicians (e.g., a state licensed physician or treatment provider) for approval.

In one or more embodiments, the clinician or physician may then conduct a telehealth consult with the patient, wherein HPI, Exam, Assessment, and/or a new treatment are created/determined. Any changes made to the original treatment request are made to the treatment, and then this modified treatment is associated with billing changes and the user/patient may be made aware of the changes. If the treatment is approved by the physician, a notification is sent to the clinician that accepted the assignment/request.

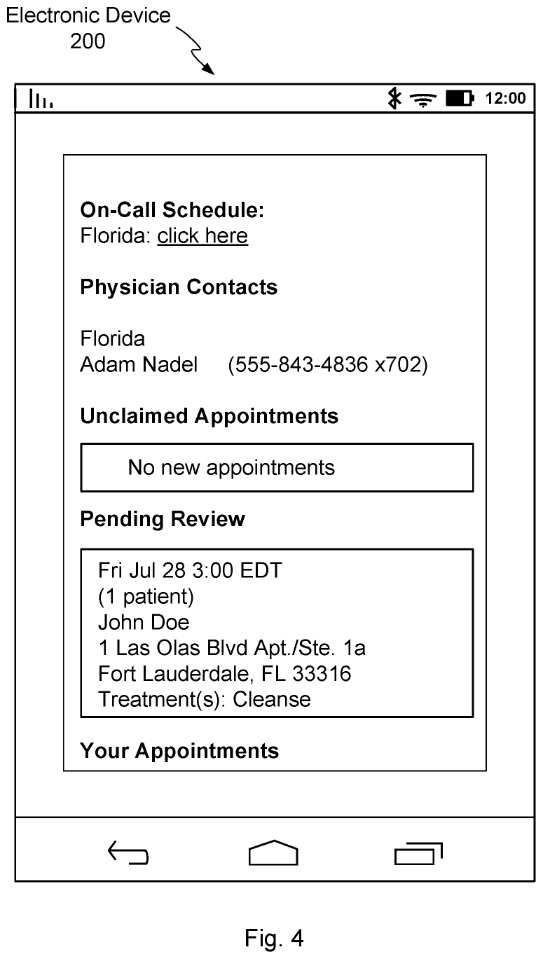

FIG. 4 shows an exemplary screenshot of a user interface presented to a clinician. After the user selects one or more health-related services of interest as depicted in FIG. 3, the communication module 130 may send a treatment request message to one or more clinicians associated with the geographic region where the service(s) are to be performed. The service request may be presented to the clinician via a user interface of the healthcare platform 100 (including the user interface module 125 and/or the communication module 130) or via another platform presented by a user interface such as a text messaging application.

FIG. 4 shows an exemplary screenshot of a user interface presented to a clinician. After the patient selects one or more health-related services of interest as depicted in FIG. 3, the communication module 130 may send a treatment request message to one or more practitioners associated with the geographic region where the service(s) are to be performed. The treatment request may be presented to the clinician via a user interface of the healthcare platform 100 (including the user interface module 125 and/or the communication module 130) or via another platform presented by a user interface such as a text messaging application.

The clinician may be presented with an option to accept or decline the pending treatment request. The option may be presented to the clinician via user interface module 125 (FIG. 1), and further via scheduling module 135 (FIG. 1). Additionally, the pending treatment request may be presented to the clinician together with other pending treatment requests from various different users of the healthcare platform 100 (FIG. 1). The different treatment requests may be presented to the clinician in a ranked list generated by the ranking module 139 (FIG. 1).

The different treatment requests may be ranked by the ranking module 139 (FIG. 1) based at least in part on identifying data associated with the user collected by the data collection module 116 (FIG. 1). The identifying data may include a history associated with a patient such as frequency/recency of previous interactions by the user with the healthcare platform 100 (FIG. 1) including past services purchased. The identifying data may also include a financial indicator associated with the patient such as an aggregate or average amount of money previously spent by the user on the healthcare platform 100 (FIG. 1). The identifying data may also include bonus pay/extra pay behavior information associated with the user. For example, the user's likelihood to provide bonus pay/extra pay, and/or the amount of bonus pay/extra pay, for the one or more services scheduled by the patient on the healthcare platform 100 (FIG. 1).

In one or more embodiments, the healthcare platform 100 includes functionality to implement surge-based compensation. In order to more quickly match clinicians with patients to thereby reduce wait times, especially during times of high treatment request demand where the supply of clinicians may not meet demand as quickly as usual, the healthcare platform 100 may implement algorithms to facilitate matching of clinicians with patients. For example, the longer a pending treatment hasn't been accepted for fulfilment by a clinician, the higher the compensation the healthcare platform 100 may offer to the clinicians to incentivize the practitioners to accept the treatment request.

In some embodiments, an algorithm of the healthcare platform 100 may then consider one or more factors to select which pending transaction to surge (e.g., whether the patient is a returning patient, what amount or in which tier the total revenue belongs in, etc.). For example, a treatment with the returning patient may take priority over all other jobs to receive a surge-based compensation adjustment. If two or more pending treatments include a returning patient, the algorithm may then consider factors related to the returning patient status like return recency, return frequency, total return count, average cost of services requested, bonus pay/extra pay behavior, or other factors to determine an order of priority.

In another example, the higher revenue job takes priority over a returning patient treatment. Or, the returning patient treatment takes priority over a higher revenue treatment, or a returning patient treatment takes priority over a higher revenue treatment if the patient has returned a number of times above a threshold within a time period or forever (e.g., above 3 returns). Or, the algorithm of the healthcare platform 100 may consider two or more criteria types concurrently. For example, both the total revenue and returning patient statuses may be considered. In one example, the higher revenue treatment takes priority over a returning patient treatment only if it meets one or more thresholds. For example, based on a relative difference in total revenue (e.g., a percentage or ratio or coefficient), based on an absolute difference (e.g., $200 or more), based on a look-up-table rule set, etc. The total revenue and returning patient statuses may be weighted differently by the algorithm.

It should be understood that multiple pending treatments may be surged concurrently. Also, that the multiple surged treatments may be surged similarly (e.g., proportionally by the same percentage or absolutely by the same amount) or disparately (e.g., some jobs surged more than others). Also, that the criteria considered by the algorithm is not limited to total revenue and returning patient status, but many other criteria like geographic proximity, services to be rendered, past patient bonus pay/extra pay statistics (e.g., amount, recency, or frequency), available supplies (e.g., medical equipment, supplements, etc.), prior engagement/relationship between the patient and practitioner, and so on.

In some embodiments, the healthcare platform 100 may apply extra pay to a clinician's compensation (may be automatically applied by the healthcare platform 100 or manually applied by a scheduling team of the healthcare platform 100). Extra pay may be defined as the minimum guaranteed amount the clinician will be paid in addition to a contractor compensation service fee

In some embodiments, extra pay is always $0 (zero) for the compensation, if not added to the treatment by the healthcare platform 100. In some embodiments, only the healthcare platform 100 can add extra pay.

In some embodiments, bonus pay (additional bonus paid to the healthcare platform 100 prior to the start of services like IV Therapy Service or ID Service) may be applied to the compensation. In some embodiments, only the greater of the extra pay or the bonus pay will be paid to the clinician.

For example, if no extra pay is added (e.g., extra pay is $0, zero dollars) and bonus pay is $50 (fifty dollars), then the clinician will receive an added $50 (fifty dollars) from the healthcare platform 100.

In another example, if extra pay is $25 (twenty-five dollars) set by the healthcare platform 100 and bonus pay is $0 (zero dollars), then the clinician will receive the extra pay of $25 (twenty-five dollars).

If extra pay is more than bonus pay, only extra pay will be added, and if bonus pay is more than extra pay, only the bonus pay will be added to the clinician compensation payment for the relevant appointment job. Extra pay and bonus pay may be subject to required service fees.

In some embodiments, extra pay may be at least in part shared between the patient and the healthcare platform 100 (e.g., from both). In one embodiment, the extra pay is guaranteed by the healthcare platform 100.

In one or more embodiments, the healthcare platform 100 requires the clinician to affirmatively indicate that they want to accept the extra pay.

In one or more embodiments, the healthcare platform 100 includes functionality to determine suggested/recommended/optimal itinerary logistics for the practitioner. For example, if there are multiple treatment request locations, the healthcare platform 100 may suggest a sequence/order to visit the locations. The healthcare platform 100 may consider such factors such as one or more clinician's schedule, the geographic locations of the treatments, returning patient status, treatment revenue amount, past patient bonus pay/extra pay amounts, available supplies and restocking locations, prior engagement/relationship between the patient and clinician, and so on. The healthcare platform 100 may consider multiple clinicians' and/or multiple patients' factors in determining to which practitioners to suggest the sequences for which patients (in other words, the best combination of pairings). The healthcare platform 100 may suggest the itinerary(ies) in place of or in concert with the surge-based compensation suggestions. For example, a repeat patient with a high total job revenue amount may be both compensation-surged and recommended as the next visit.

After the clinician (also referred to as a practitioner) accepts a pending service request, multiple parties may be notified with confirmation of the acceptance. For example, the clinician, physician, an admin of the healthcare platform 100, and the user may all be notified of the clinician's acceptance of the pending service request. In the event that there is a mismatch between the clinician and physician, the admin of the healthcare platform 100 may be notified.

FIG. 5 shows an exemplary screenshot of a user interface presented to a physician. For example, a physician may receive a notification that a treatment request is ready to be approved (e.g., after a clinician has accepted the treatment request). The physician may be shown certain information about a patient such as their name and age, or additional health information from an EMR or sent by the patient (in some cases in response to questions sent to a patient). A physician may then approve a treatment by signing their name and engaging a submission user interface element, or via an alternative method.

The clinician may then, at the scheduled time for the healthcare-related service, meet the user (e.g., patient) at their specified choice of location to carry out the requested treatment(s). Prior to performing any treatment, the clinician may review a portion of the patient's EMR data and/or information submitted with the patient's treatment request. For example, a physician may review the submitted treatment request information and add further notes, thereafter a clinician may review the submitted treatment request information and the notes added by the physician. Instead of rewriting/repeating the same or similar notes as the physician, the clinician may only be required to indicate that they have reviewed the notes and agree with the notes. If there is a disagreement or discrepancy between understandings of the notes (e.g., between the patient and physician, the physician and the clinician, or between any other providers), the healthcare platform 100 may flag or issue an alert to relevant participants of the healthcare platform 100 for investigation.

Prior to performing any service, the clinician may verify a number of different vitals and patient information. These vitals and patient information may again be transmitted to the physician or checked against previous data approved by the physician. If there is a disagreement or discrepancy between the notes and the recently on-site measured vitals (or the vitals include causes of concern), the healthcare platform 100 may flag or issue an alert to relevant participants of the healthcare platform 100 for investigation. After all vital and patient related information is verified, the clinician may perform the requested treatment(s) for the user.

In one or more embodiments, the healthcare platform 100 includes functionality to provide real-time service cost information to the clinician and/or patient. For example, traditional healthcare environments silo the practitioner from the costs/billing associated with the services, where the patient learns of the costs/billing only after they've been incurred. However, the healthcare platform 100 allows the clinician and/or patient to see and consider costs associated with additions to, removals from, or modifications to the services to be rendered.

Upon completion, the clinician may receive all or a part of the service fee determined by the pricing module (described herein).

It should be appreciated that instead of the workflow discussed above, other workflows are possible. For example, in some embodiments, a telehealth consult first occurs with a physician, which, as discussed herein, may include a state-licensed physician/doctor, physician assistant, or nurse practitioner. If deemed appropriate, based on the plan clinician and/or physician entered into the healthcare platform 100, a request for at-home (or location) care is made. The clinician may indicate an ASAP status or specific time/date. This will trigger a notification for a nurse, or any provider (including physicians) to see and treat the user/patient.

Alternatively, if the clinician and/or physician's plan allows for the patient to receive treatment, and no date is determined, the patient may log into the healthcare platform 100 and select their own date/time for treatment/appointment. This will then trigger a notification for a nurse or any provider (including physicians) to see and treat the user/patient.

In one or more embodiments, the healthcare platform 100 includes functionality to monitor and record the geolocation of the clinician via location services of a corresponding electronic device (e.g., a cellular phone or tablet). The geolocation may be determined via GPS, cellular tower triangulation, WiFi positioning/triangulation, IP address location associations, Bluetooth, NFC, or other technologies capable of aiding in location determination.

The healthcare platform 100 may use the geolocation information to confirm that the clinician visited the service location, the time the clinician arrived, and/or the time the clinician departed. The patient's geolocation can also be recorded for cross-checking with the clinician geolocation info (e.g., to determine whether both parties were in the same area on time). Such information may be used for extra pay determinations, clinician performance metrics, and so on (e.g., a clinician may be compensated better when they arrive at a treatment location on time). In some cases, the attributes of the patient (e.g., if the patient is a return customer), may determine an amount that a clinician is compensated for a treatment in combination with the timeliness of the clinician. The geolocation and/or timestamp may be included in the consent form to be signed by the patient or the job receipt (e.g., for aiding in dispute resolutions).

In one or more embodiments, the healthcare platform 100 includes functionality to automatically check in/follow up with the patient after the services have been rendered (e.g., a message via the user interface module 125 and/or an SMS text via the communication module 130). The check in may confirm that the patient received the service, determine the health status of the patient, confirm that the patient is satisfied with the service, confirm that the patient is following through with any prescribed instructions, and/or confirm that the patient is in good health.

In some embodiments, the healthcare platform 100 may check in according to a predefined schedule (e.g., 1 hour after, the evening of the same day, at 9 am the following day, etc.). In some embodiments, the healthcare platform 100 may check in according to algorithmic determinations (e.g., sooner or later based on the patient's HPI, particular services rendered, likelihood of patient wake state based on age and time zone, etc.).

In one or more embodiments, the healthcare platform 100 includes functionality to manage inventory (e.g., equipment/supplies), ordering, distribution, authorized use of equipment/supplies (e.g., needles, supplements, pharmaceuticals, etc.), theft detection, and other purposes. For example, healthcare supplies may be stored in one or more facilities in a geographic region where healthcare services are rendered, such as a warehouse, health clinic, or hospital. Based on the healthcare supply needs of requested jobs, the healthcare platform 100 may confirm availability of requisite healthcare supplies, and order or plan to order, replacement supplies ahead of stock outages. In some embodiments, clinicians may not receive a treatment request if they do not have the requisite equipment/supplies.

The supplies may include identifiers (e.g., serial numbers, barcodes, QR codes, NFC tags, etc.) that are scannable via an electronic device of a clinician or supporting member to facilitate identification of the correct supply, and confirmation that the correct supply has been collected by the clinician. In one or more embodiments, the healthcare platform 100 includes functionality to require scanning of at least some of the supplies at the care location to confirm the use of the correct supplies and to further confirm inventory. The healthcare platform 100 may rely on location services and timestamp to further confirm that the supplies are being used at the proper place and time.

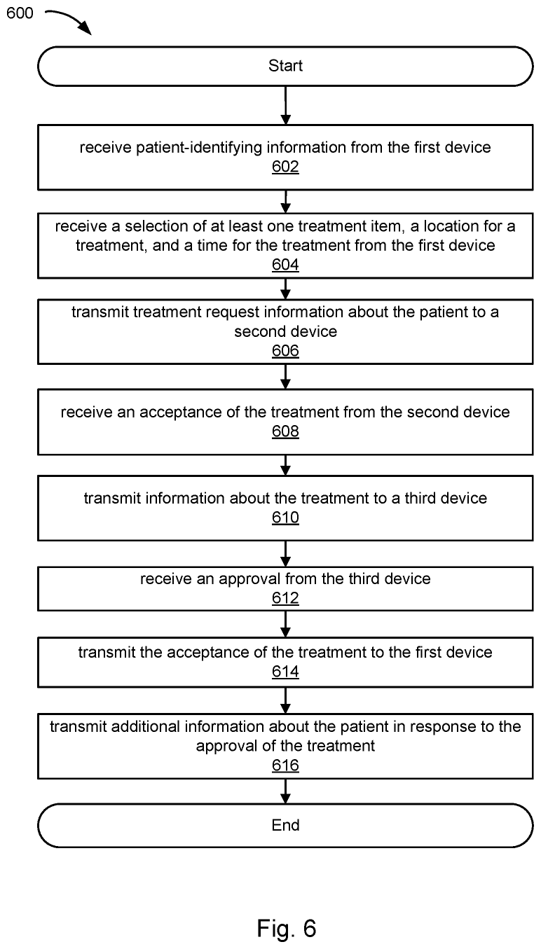

FIG. 6 shows a flowchart 600 of a method for providing a treatment. While the various steps in this flowchart are presented and described sequentially, one of ordinary skill will appreciate that some or all of the steps can be executed in different orders and some or all of the steps can be executed in parallel. Further, in one or more embodiments, one or more of the steps described below can be omitted, repeated, and/or performed in a different order. Accordingly, the specific arrangement of steps shown in FIG. 6 should not be construed as limiting the scope of the invention.

At STEP 602, patient-identifying information is received from a first device. For example, a patient may sign into the healthcare platform and send identifying information from their smart phone. This information could be an account ID, a name, a date of birth, a phone number, etc. In various embodiments, this identifying information is used to access a patient's electronic health records, which the health platform may later provide to a clinician and a physician.

Once a patient is logged into the healthcare platform, they may choose one or more treatments. Treatments may include, for example, in-home IV hydration and vitamins.

In some embodiments, health-related questions may be transmitted to the first device. For example, the healthcare platform may send a patient questions about their allergies, what type of food they've ingested, a pain level, etc. The health platform may receive health-related questions, and in turn recommend a treatment to a patient based on answers to those health-related questions. Also, in some embodiments, a health platform may recommend a treatment based on the price of a particular treatment or portion thereof. Further, a health platform may recommend a treatment based on a combination of attributes of a patient and the price of a treatment.

It should be understood that in various embodiments, a first electronic device may be logged into a patient account, a second electronic device may be logged into a clinician account, and/or a third electronic device may be logged into a physician account. In some embodiments, there may be additional account types, such as one for a health plan or an administrator.

In some embodiments, an account may have a balance associated with it. For example, a patient account may have an amount the patient owes or has loaded onto their account. Similarly, a clinician or a physician may have an amount associated with their account (e.g., that they've earned from approving or providing treatments), which they can transfer from the health platform to a bank account.

In some embodiments, a healthcare platform may determine a demand for services (e.g., based on an amount of available clinicians and an amount of treatment requests). Based on the demand for services, the healthcare platform may alter the cost of treatments by making them more or less expensive for the patient (this is sometimes referred to as surge pricing as described above). Also, an amount that the healthcare platform pays (e.g., adds receivables to a clinician or physician's account) may be increased or decreased based on the demand for services.

Various embodiments of the present disclosure include systems and methods for displaying a price of a treatment to a user before they “checkout” (e.g., submit their request for treatment). When a patient checks out, they can see the complete amount of their treatments, as opposed to charging the patient's insurance company at a later time. A complete amount may include the cost of clinician time (which may be based on a metro area), materials, the cost of physician time, any additional costs due to demand (e.g., surge pricing), any tip, any bonus pay, or any combination thereof. In some embodiments, after placing an order a physician may recommend modifying a treatment (e.g., adding vitamins), in which case a message may be sent to a patient asking them if they agree to a different price. The patient may accept or reject the modified treatment and increase or decrease the amount they pay to the healthcare platform.

At STEP 604, a selection/request of at least one treatment item, a location for a treatment, and a time for the treatment is received from the first device. For example, the patient may select IV Hydration and a type of vitamins, their home or office, and a time for a clinician to provide the treatment. This information is sent to the healthcare platform, and a variety of things may happen.

In some embodiments, a variety of attributes about the patient and the treatment may be used to determine which clinician the treatment request is sent to. For example, the desired location for the treatment may be used to determine which clinicians to send the treatment request to. In some embodiments, information about a patient's previous tips may be used to determine which clinicians to send the treatment request to. Further, in some embodiments, which clinician to send a treatment request to may be based on whether the treatment is for acute care (e.g., a headache), lab-based services, and/or wellness services. A patient may be classified as needing acute care, lab-based services, and/or wellness services and a particular clinician may receive a treatment request for that patient based on whether their attributes such as whether they are qualified to perform acute care (or a specific type of acute care).

In some embodiments, which clinicians to send a treatment request to may depend on the equipment/supplies a clinician has. For example, the healthcare platform may include a database that indicates what equipment/supplies a clinician has with them on a particular day. The healthcare platform may also have the ability to track what equipment/supplies a clinician takes from a repository of equipment/supplies, such as a warehouse, clinic, or hospital.

At STEP 606, treatment request information about the patient is transmitted to a second device. For example, a treatment request may be sent to a clinician's smart phone, and the clinician may accept the treatment request to go to the patient's home to provide the treatment.

As described above, a clinician may be a nurse that is an independent contractor, a member of a nursing agency, etc. In some cases, a clinician may be a person other than a nurse that is licensed to provide treatments. For example, a clinician may be a certified dialysis technician, a surgical technician, etc.

In some embodiments, clinicians may only receive a subset of potential requests sent to their devices/accounts. For example, a clinician may only receive a treatment request if they have a particular specialty, such as outpatient oncology, rehabilitation, community health, pediatric rehab, etc. As other examples, clinicians may only receive particular treatment requests if they have a rating above a certain threshold, if they have an hourly rate above or below a certain amount, if they have a certain amount of experience, if they have seen a particular patient before, if they are in a certain proximity of the location where the treatment is desired, etc.

In some embodiments, a clinician may send information to the healthcare platform indicating what type of patients they would like to receive treatment requests from. For example, a clinician may select a certain geographic area within which they are willing to provide treatments. As additional examples, clinicians may configure their accounts such that they only receive treatment requests from patients that require a particular type of care (e.g., only IV Hydration patients), patients that tip a certain amount on average, patients that have ratings above a certain threshold, patients that are a certain gender or age, etc.

At STEP 608, an acceptance of the treatment is received from a second device. For example, a clinician may accept one or more of the treatment requests from one or more patients from a list of potential patients and/or treatments shown on their smart phone. In some embodiments, the clinician's device may display the treatment requests based on the distance between them and the desired location of the treatment, whether a patient has a certain attribute (e.g., whether the patient is a preferred patient (that either tips a certain amount on average or is a repeat patient)), an estimated amount of time the patient's treatment will take, etc.

In some embodiments, a clinician may receive information associated with what equipment/supplies they have. For example, the clinician's device may indicate that they are missing certain equipment/supplies. The device may indicate where the nearest equipment/supply repository is (e.g., warehouse), and/or show a clinician what treatment requests they could accept if they were to have certain additional equipment/supplies.

At STEP 610, information about the treatment is transmitted to a third device. For example, after a clinician has accepted a treatment request a message may be sent to a physician's phone, tablet, or computer with information about the patient and the requested treatment. A physician may receive patient-identifying information, including information from an EHR, EMR, etc. which may help the physician determine whether to approve a requested treatment.

This application refers to physicians as approving the treatment requests that were accepted by clinicians. However, it should be understood that a user that is not a physician (e.g., a therapist, a nurse) could approve the treatment request accepted by a clinician.

Which physician is sent the request for the approval of a treatment request may be based on a variety of attributes associated with the patient, clinician, or physician. For example, the physician that receives a request for a treatment approval may be based on that physician's specialties, or whether they approved treatment requests for the particular patient before. Similarly, the physician that receives a request for an approval of a treatment may be selected based on whether they have worked with the clinician that accepted the treatment request before.

In some embodiments, a physician may suggest a different treatment for a patient. For example, a physician may see that a patient has requested a treatment that only included IV Hydration, and suggest a treatment that includes vitamins. This may be sent to the patient via the healthcare platform, along with the price of the new suggested treatment. In some cases, the physician sees whether the patient accepts the new suggested treatment and may be required to provide additional approval, while in other cases the new suggested treatment is assumed to be approved by the physician and the physician does not need to approve the modified treatment.

At STEP 612, approval is received from the third device. For example, the physician may approve a treatment request that has been approved by a clinician. As described above, the physician may suggest a different treatment. In some cases that the cost to the patient of the suggested new treatment will be shown to the physician (i.e., transmitted from the health platform to the third device).

At STEP 614, acceptance of the treatment is transmitted to the first device. For example, after the requested treatment has been approved by a physician, the patient may be sent a message indicating that the treatment has been approved. In some embodiments, a clinician will also receive a message indicating the treatment has been approved by the physician.

In some cases, after acceptance by a clinician and approval by a physician a patient may receive a confirmation message (e.g., in an app or via email) indicating at least the time of the treatment, the location of the treatment, the type of treatment, the equipment/supplies that will be used during the treatment, and/or an estimated time that the treatment will take. The message may also include the identity of the clinician and the physician, their respective ratings, and/or their respective specialties.

In some embodiments, a healthcare platform may send a message to a patient indicating that their treatment request has been accepted, but suggest that the treatment occur at a different time or location. For example, a clinician may accept a request but indicate that they can only perform the treatment if it is at a particular time or within a certain geographic area. In such a case, for example, a patient may be asked whether they accept the new time before a confirmation message is sent to the patient.

At STEP 616, additional information about the patient is transmitted in response to the approval of the treatment. For example, after a physician approves a treatment, a clinician may receive additional health information about the patient that requested the treatment. For example, after a physician approves a treatment, the clinician may receive information about what type of treatment is sought, a requested location for a treatment, and/or a patient's name, date of birth, allergies, and/or gender.

In some embodiments, information about a second patient may be transmitted to the second device. For example, information about a second patient may be sent to a clinician, wherein the second patient is determined based at least in part on the location of the clinician, the location of the first patient, and/or the location of the second patient. In some embodiments, in response to a clinician accepting a treatment request from a particular patient, the clinician may be presented with an additional treatment request from another patient in the same area. The additional treatment request may also be based on the time of a first treatment and the requested time of another treatment. For example, a healthcare platform may suggest a treatment request to a clinician in response to the clinician having a first treatment at a first time (e.g., 10:00 a.m.) and the requested time of another treatment (which may be within a certain distance from the first treatment) being a threshold amount of time after the first treatment time (e.g., 1:00 p.m.).

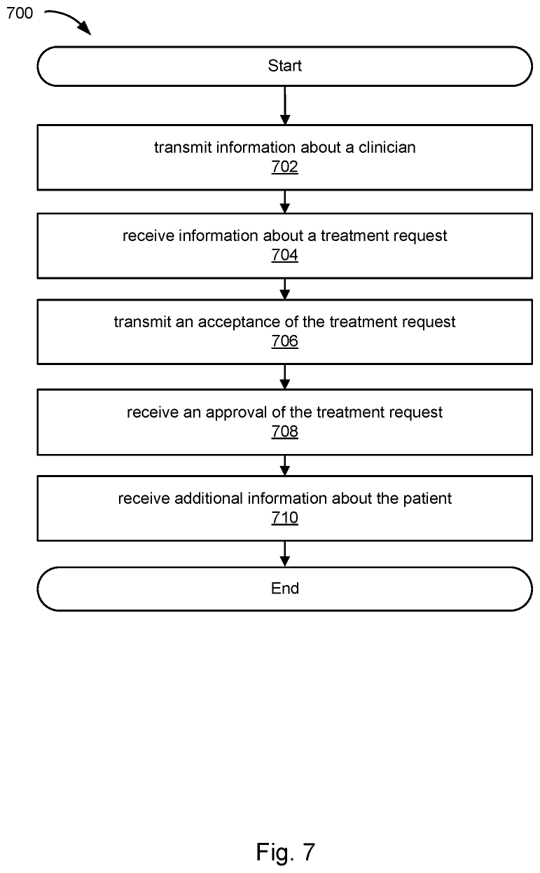

FIG. 7 shows a flowchart 700 of a method for providing a treatment. While the various steps in this flowchart are presented and described sequentially, one of ordinary skill will appreciate that some or all of the steps can be executed in different orders and some or all of the steps can be executed in parallel. Further, in one or more embodiments, one or more of the steps described below can be omitted, repeated, and/or performed in a different order. Accordingly, the specific arrangement of steps shown in FIG. 7 should not be construed as limiting the scope of the invention.

At STEP 702, information about a clinician is transmitted. For example, an electronic device associated with a clinician may log into a healthcare platform, and information associated with that clinician may be transmitted to the healthcare platform. In some embodiments, a clinician may be associated with a rating (also referred to as a ranking), which may determine whether a clinician receives certain requests for treatments from various patients. Such a rating may be based on reviews of the clinician by patients, or by the clinician's history of arriving on time (e.g., at the scheduled time of the treatment). If a clinician is threshold amount of time late to a threshold amount of treatments (e.g., if the clinician is over ten minutes late three or more times), then the healthcare platform may rate the clinician lower and/or present the clinician with a certain subset of treatment requests that the clinician can accept. Similarly, a clinician may have a lower rating and/or only receive a certain subset of treatment requests if their average amount of time being late for a treatment is above a certain amount.

In some embodiments, information gathered from a clinician's equipment/supplies may be used by a healthcare platform. For example, a location of a clinician's electronic device may be used by a healthcare platform to determine whether a clinician arrived at a scheduled treatment on time. In some embodiments, the location of a patient's device and the location of a clinician's device may be used to determine whether a clinician arrived to an appointment on time, when the clinician left the treatment, or other information. Other equipment, such as a pump or other IV-related equipment may be used to determine when and whether a treatment was performed (e.g., the device may have cellular connectivity).

At STEP 704, information is received about a treatment request. For example, a clinician's electronic device may receive one or more treatment requests from one or more patients. A healthcare platform may provide the clinician with requests for treatments from certain patients, requests for treatments from patients in certain locations, requests for treatments based on the clinician's schedule (which may be stored by the healthcare platform), etc.

At STEP 706, an acceptance of the treatment request is transmitted. For example, a clinician may accept one or more treatments that were requested by patients, and that acceptance may be transmitted to a healthcare platform. In some embodiments, a clinician may choose a treatment and send a message requesting a different time.

At STEP 708, an approval of a treatment request is received. For example, in response to a clinician accepting a treatment request a request for approval is sent to a physician. In response to the treatment request being approved by a physician, the clinician (e.g., at their electronic device) may receive a notice indicating the treatment has been approved by a physician

At STEP 710, additional information about a patient may be received. For example, a clinician may receive information about the patient such as their location, name, age, gender, etc.

Embodiments described herein may be discussed in the general context of computer-executable instructions residing on some form of computer-readable storage medium, such as program modules, executed by one or more computers or other devices. By way of example, and not limitation, computer-readable storage media may comprise non-transitory computer-readable storage media and communication media; non-transitory computer-readable media include all computer-readable media except for a transitory, propagating signal. Generally, program modules include routines, programs, objects, components, data structures, etc., that perform particular tasks or implement particular abstract data types. The functionality of the program modules may be combined or distributed as desired in various embodiments.

Computer storage media includes volatile and nonvolatile, removable and non-removable media implemented in any method or technology for storage of information such as computer-readable instructions, data structures, program modules or other data. Computer storage media includes, but is not limited to, random access memory (RAM), read only memory (ROM), electrically erasable programmable ROM (EEPROM), flash memory or other memory technology, compact disk ROM (CD-ROM), digital versatile disks (DVDs) or other optical storage, magnetic cassettes, magnetic tape, magnetic disk storage or other magnetic storage devices, or any other medium that can be used to store the desired information and that can be accessed to retrieve that information.

Communication media can embody computer-executable instructions, data structures, and program modules, and includes any information delivery media. By way of example, and not limitation, communication media includes wired media such as a wired network or direct-wired connection, and wireless media such as acoustic, radio frequency (RF), infrared, and other wireless media. Combinations of any of the above can also be included within the scope of computer-readable media.

Embodiments may be implemented on a specialized computer system. The specialized computing system can include one or more modified mobile devices (e.g., laptop computer, smart phone, personal digital assistant, tablet computer, or other mobile device), desktop computers, servers, blades in a server chassis, or any other type of computing device(s) that include at least the minimum processing power, memory, and input and output device(s) to perform one or more embodiments.

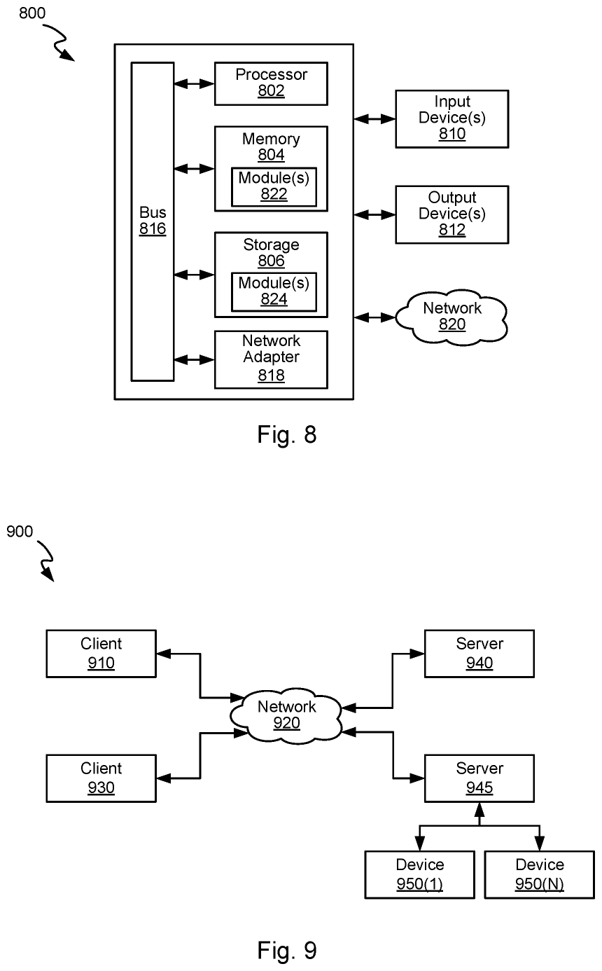

For example, as shown in FIG. 8, the computing system 800 may include one or more computer processor(s) 802, associated memory 804 (e.g., random access memory (RAM), cache memory, flash memory, etc.), one or more storage device(s) 806 (e.g., a hard disk, an optical drive such as a compact disk (CD) drive or digital versatile disk (DVD) drive, a flash memory stick, etc.), a bus 816, and numerous other elements and functionalities. The computer processor(s) 802 may be an integrated circuit for processing instructions. For example, the computer processor(s) may be one or more cores or micro-cores of a processor.

In one or more embodiments, the computer processor(s) 802 may be an integrated circuit for processing instructions. For example, the computer processor(s) 802 may be one or more cores or micro-cores of a processor. The computer processor(s) 802 can implement/execute software modules stored by computing system 800, such as module(s) 822 stored in memory 804 or module(s) 824 stored in storage 806. For example, one or more of the modules described herein can be stored in memory 804 or storage 806, where they can be accessed and processed by the computer processor 802. In one or more embodiments, the computer processor(s) 802 can be a special-purpose processor where software instructions are incorporated into the actual processor design.

The computing system 800 may also include one or more input device(s) 810, such as a touchscreen, keyboard, mouse, microphone, touchpad, electronic pen, or any other type of input device. Further, the computing system 800 may include one or more output device(s) 812, such as a screen (e.g., a liquid crystal display (LCD), a plasma display, touchscreen, or other display device), a printer, external storage, or any other output device. The computing system 800 may be connected to a network 820 (e.g., a local area network (LAN), a wide area network (WAN) such as the Internet, mobile network, or any other type of network) via a network interface connection 818. The input and output device(s) may be locally or remotely connected (e.g., via the network 820) to the computer processor(s) 602, memory 604, and storage device(s) 806.

One or more elements of the aforementioned computing system 800 may be located at a remote location and connected to the other elements over a network 820. Further, embodiments may be implemented on a distributed system having a plurality of nodes, where each portion may be located on a subset of nodes within the distributed system. In one embodiment, the node corresponds to a distinct computing device. Alternatively, the node may correspond to a computer processor with associated physical memory. The node may alternatively correspond to a computer processor or micro-core of a computer processor with shared memory and/or resources.

For example, one or more of the software modules disclosed herein may be implemented in a cloud computing environment. Cloud computing environments may provide various services and applications via the Internet. These cloud-based services (e.g., software as a service, platform as a service, infrastructure as a service, etc.) may be accessible through a Web browser or other remote interface.

One or more elements of the above-described systems may also be implemented using software modules that perform certain tasks. These software modules may include script, batch, routines, programs, objects, components, data structures, or other executable files that may be stored on a computer-readable storage medium or in a computing system. These software modules may configure a computing system to perform one or more of the example embodiments disclosed herein. The functionality of the software modules may be combined or distributed as desired in various embodiments. The computer readable program code can be stored, temporarily or permanently, on one or more non-transitory computer readable storage media. The non-transitory computer readable storage media are executable by one or more computer processors to perform the functionality of one or more components of the above-described systems and/or flowcharts. Examples of non-transitory computer-readable media can include, but are not limited to, compact discs (CDs), flash memory, solid state drives, random access memory (RAM), read only memory (ROM), electrically erasable programmable ROM (EEPROM), digital versatile disks (DVDs) or other optical storage, and any other computer-readable media excluding transitory, propagating signals.

FIG. 9 is a block diagram of an example of a network architecture 700 in which client systems 910 and 930, and servers 940 and 945, may be coupled to a network 920. Network 920 may be the same as or similar to network 820. Client systems 910 and 930 generally represent any type or form of computing device or system, such as client devices (e.g., portable computers, smart phones, tablets, smart TVs, etc.).

Similarly, servers 940 and 945 generally represent computing devices or systems, such as application servers or database servers, configured to provide various database services and/or run certain software applications. Network 920 generally represents any telecommunication or computer network including, for example, an intranet, a wide area network (WAN), a local area network (LAN), a personal area network (PAN), or the Internet.

With reference to computing system 800 of FIG. 8, a communication interface, such as network adapter 818, may be used to provide connectivity between each client system 910 and 930, and network 920. Client systems 910 and 930 may be able to access information on server 940 or 945 using, for example, a Web browser, thin client application, or other client software. Such software may allow client systems 910 and 930 to access data hosted by server 940, server 945, or storage devices 950(1)-(N). Although FIG. 9 depicts the use of a network (such as the Internet) for exchanging data, the embodiments described herein are not limited to the Internet or any particular network-based environment.

In one embodiment, all or a portion of one or more of the example embodiments disclosed herein are encoded as a computer program and loaded onto and executed by server 940, server 945, storage devices 950(1)-(N), or any combination thereof. All or a portion of one or more of the example embodiments disclosed herein may also be encoded as a computer program, stored in server 940, run by server 945, and distributed to client systems 910 and 930 over network 920.

Although components of one or more systems disclosed herein may be depicted as being directly communicatively coupled to one another, this is not necessarily the case. For example, one or more of the components may be communicatively coupled via a distributed computing system, a cloud computing system, or a networked computer system communicating via the Internet.

And although only one computer system may be depicted herein, it should be appreciated that this one computer system may represent many computer systems, arranged in a central or distributed fashion. For example, such computer systems may be organized as a central cloud and/or may be distributed geographically or logically to edges of a system such as a content/data delivery network or other arrangement. It is understood that virtually any number of intermediary networking devices, such as switches, routers, servers, etc., may be used to facilitate communication.

While the invention has been described with respect to a limited number of embodiments, those skilled in the art, having benefit of this disclosure, will appreciate that other embodiments may be devised that do not depart from the scope of the invention as disclosed herein.

While the present disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered as examples because other architectures can be implemented to achieve the same functionality.

The process parameters and sequence of steps described and/or illustrated herein are given by way of example only. For example, while the steps illustrated and/or described herein may be shown or discussed in a particular order, these steps do not necessarily need to be performed in the order illustrated or discussed. Some of the steps may be performed simultaneously. For example, in certain circumstances, multitasking and parallel processing may be advantageous. The various example methods described and/or illustrated herein may also omit one or more of the steps described or illustrated herein or include additional steps in addition to those disclosed.

It is understood that a “set” can include one or more elements. It is also understood that a “subset” of the set may be a set of which all the elements are contained in the set. In other words, the subset can include fewer elements than the set or all the elements of the set (i.e., the subset can be the same as the set).

What is claimed is:

1. A method, comprising:
receiving, from a first electronic device, patient-identifying information, wherein the patient-identifying information includes a name, a date of birth, and a phone number;
receiving, from the first electronic device, a selection of at least one treatment, a location for the treatment, and a time for the treatment;
transmitting, to a second electronic device, treatment request information about the patient, wherein the treatment request information includes the location for a treatment, electronic health records associated with the patient, and the at least one treatment, and wherein the second electronic device is associated with a clinician;
receiving, from the second electronic device, an acceptance of the treatment,
transmitting, to a third electronic device, information about the treatment, wherein the third electronic device is associated with a physician;
receiving, from the third electronic device, an approval of the treatment;
transmitting, to the first electronic device, the acceptance of the treatment; and
transmitting, to the second electronic device, additional information about the patient in response to the approval of the treatment.

2. The method of claim 1, wherein the first electronic device is logged into a patient account, the second electronic device is logged into a clinician account, and the third electronic device is logged into a physician account.

3. The method of claim 1, further comprising:
receiving, from the first electronic device, an amount to tip the clinician.

4. The method of claim 1, further comprising:
increasing a payment to the clinician based at least in part on the number of pending treatments.

5. The method of claim 1, further comprising:
transmitting, to the first electronic device, treatment-cost information before the transmission of the treatment request information about the patient to the second electronic device.

6. The method of claim 5 wherein the cost information is the total amount the patient will be charged for the at least one treatment, and does not include an amount to tip the clinician.

7. The method of claim 5, further comprising:
incentivizing the provider to approve the treatment at the cost associated with the treatment.

8. The method of claim 1, further comprising:
transmitting, to the second device, treatment information about a second patient, wherein the second patient is determined based on the location of the clinician and the location of the second patient.

9. The method of claim 1, further comprising:
transmitting, to the first electronic device, one or more health-related questions;
receiving, from the first device, responses to the one or more health-related questions; and
transmitting a recommendation for a treatment based on the one or more health-related questions.

10. The method of claim 1, wherein the treatment request information includes a location associated with the patient.

11. The method of claim 1 wherein the patient-identifying information includes one or more of: a date of birth, a social security number, and a name.

12. A system for scheduling treatments, the system comprising:
a computer processor;
a memory; and
a treatment scheduling engine executing on the computer processor and configured to:
receive, from a first electronic device, patient-identifying information, wherein the patient-identifying information includes a name, a date of birth, and a phone number;
receive, from the first electronic device, a selection of at least one treatment, a location of the treatment, and a time for the treatment;
transmit, to a second electronic device, treatment request information about the patient, wherein the treatment request information includes the location for a treatment, electronic health records associated with the patient, and the at least one treatment, and wherein the second electronic device is associated with a clinician;
receive, from the second electronic device, an acceptance of the treatment;
transmit, to a third electronic device, information about the treatment, wherein the third electronic device is associated with a physician;
receive, from the third electronic device, an approval of the treatment;
transmit, to the first electronic device, the acceptance of the treatment; and
transmit, to the second electronic device, additional information about the patient in response to the approval of the treatment.

13. The system of claim 12, wherein the first electronic device is logged into a patient account, the second electronic device is logged into a clinician account, and the third electronic device is logged into a physician account.

14. The system of claim 12, wherein the treatment scheduling engine executing on the computer processor is further configured to:
receive, from the first electronic device, an amount to tip the clinician.

15. The system of claim 12, wherein the treatment scheduling engine executing on the computer processor is further configured to:
increase a payment to the clinician based at least in part on the number of pending treatments.

16. The system of claim 12, wherein the treatment scheduling engine executing on the computer processor is further configured to:
transmit, to the first electronic device, treatment-cost information before the transmission of the treatment request information about the patient to the second electronic device.

17. The system of claim 16, wherein the cost information is the total amount the patient will be charged for the at least one treatment, and does not include an amount to tip the clinician.

18. The system of claim 16, wherein the treatment scheduling engine executing on the computer processor is further configured to:
incentivize the provider to approve the treatment at the cost associated with the treatment.

19. The system of claim 12, wherein the treatment scheduling engine executing on the computer processor is further configured to:
transmit, to the second device, treatment information about a second location, wherein the second patient is determined based on the location of the clinician and the location of the second patient.

20. The system of claim 12, wherein the treatment scheduling engine executing on the computer processor is further configured to:
transmit, to the first electronic device, one or more health-related questions;
receive, from the first device, responses to the one or more health-related questions; and
transmit a recommendation for a treatment based on the one or more health-related questions.
